SEQUENCE LISTING (6 sequences)

SEQ ID NO: 1 - RNA, 21 nt, Artificial Sequence
  Description of Artificial Sequence  siRNA directed to ATR nucleic acid
ccuccgugauguugcuugauu

SEQ ID NO: 2 - RNA, 21 nt, Artificial Sequence
  Description of Artificial Sequence  siRNA directed to ATR nucleic acid
gccaugagcgcaaaggcaguu

SEQ ID NO: 3 - RNA, 21 nt, Artificial Sequence
  Description of Artificial Sequence  siRNA directed to Rad17 nucleic acid
cagacuggguugacccaucuu

SEQ ID NO: 4 - DNA, 21 bp, Artificial Sequence
  Description of Artificial Sequence  fragment of Homo sapiens ATR
aacctccgtgatgttgcttga

SEQ ID NO: 5 - DNA, 21 bp, Artificial Sequence
  Description of Artificial Sequence  fragment of Homo sapiens ATR
aagccatgagcgcaaaggcag

SEQ ID NO: 6 - DNA, 21 bp, Artificial Sequence
  Description of Artificial Sequence  fragment of Homo sapiens Rad17
aacagactgggttgacccatc

(19) United States
(12) Patent Application Publication
(10) Pub. No.: US 2007/0134758 A1
(43) Pub. Date: Jun. 14, 2007

(54) Methods of inhibiting hiv-1 vpr activity and hiv-1 infectivity using atr or rad17 inhibitors

(76) Inventors: Vicente Planelles, Salt Lake City, UT (US); Mikhail Roshal, Rochester, NY (US); Yong Hong Zhu, Rochester, NY (US)

Correspondence Address: Edwin V Merkel;Nixon Peabody, Clinton Square, P O Box 31051, Rochester, NY, 14603-1051, US

(21) Appl. No.: 10/504,249

(22) PCT Filed: Feb. 13, 2003
(86) PCT No.: PCT/US03/04400

§ 371 Date: Jul. 19, 2005

Related U.S. Application Data
(60) Provisional application No. 60/357,159, filed on Feb. 13, 2002.

(51) Int. Cl. C12Q 1/68; C12P 21/06

(52) U.S. Cl. 435/69.1; 435/6

(57) ABSTRACT

Methods are disclosed for inhibiting the activity of a viral protein R (Vpr), inhibiting HIV replication, and treating or preventing an HIV infection through the use of an inhibitor of ATR or Rad17. Newly discovered ATR or Rad17 inhibitors are disclosed for use in accordance with the present invention, as are previously known compounds based upon their newly discovered property as inhibitors of ATR or Rad17.

The present application is entitled to the priority benefit of U.S. Provisional Patent Application Ser. No. 60/357,159 filed Feb. 13, 2002, which is hereby incorporated by reference in its entirety.

### STATEMENT OF GOVERNMENT INTEREST

The present invention was made, at least in part, with funding received from the National Institutes of Health Grant Nos. R01AI49057 and R21AI054188. The U.S. government may retain certain rights in this invention.

### FIELD OF THE INVENTION

The present invention relates to the use of inhibitors of the ATM- and Rad3-related protein, designated ATR, and inhibitors of Rad17 for inhibiting the activity of the HIV-1 viral protein R (Vpr) and for inhibiting HIV-1 infectivity of cells in which ATR or Rad17 has been inhibited.

### BACKGROUND OF THE INVENTION

DNA damage-signaling pathways consist of a network of interacting and occasionally redundant signals that may lead to the inactivation of the Cdc2/CyclinB complex (O'Connell et al., “The G2-Phase DNA-Damage Checkpoint,” Trends Cell Biol. 10:296-303 (2000); Ohi et al., “Regulating the Onset of Mitosis,” Curr Opin Cell Biol. 11:267-273 (1999); Smits et al., “Checking Out the G(2)/M Transition,” Biochim Biophys Acta. 1519:1-12 (2001); Walworth, N. C., “Cell-Cycle Checkpoint Kinases: Checking in on the Cell Cycle,” Curr Opin Cell Biol. 12:697-704 (2000); Zhou et al., “The DNA Damage Response: Putting Checkpoints in Perspective,” Nature 408:433-439 (2000)) and cell cycle arrest in G2. A major point of regulation of the Cdc2/CyclinB cyclin complex is through inhibitory phosphorylation of Cdc2 on Tyr-15. Phosphorylation of the adjacent residue, Thr-14, also contributes to the inhibition of Cdc2 activity. Cdc25C is a dual specificity phosphatase that dephosphorylates Cdc2 on both Tyr-15 and Thr-14, leading to Cdc2 activation. Upon induction of the DNA-damage checkpoint, Cdc25C is inactivated through the actions of several kinases, including Chk1 and Chk2, which are under the control of the phosphatidyl inositol-3 kinase (PI3K)-like proteins, ATR and ATM.

ATR and ATM control the induction of the DNA damage checkpoint by responding to a variety of DNA-damaging agent-induced abnormal DNA structures (Westphal, C. H., “Cell-Cycle Signaling: ATM Displays its Many Talents,” Curr Biol. 7:R789-792 (1997)). Their roles are partially redundant, but with some important distinctions, both with regards to substrate preference and the types of the DNA damage to which the kinases respond. In response to cytotoxic stress ATM is responsible for phosphorylation of the Chk2 protein kinase, while ATR phosphorylates Chk1. ATR is primarily responsible for enforcement of the cell cycle arrest checkpoint in response to intra S phase cytotoxic stress (Cliby et al., “S Phase and G2 Arrests Induced by Topoisomerase I Poisons are Dependent on ATR Kinase Function,” J. Biol. Chem. 277:1599-1606 (2002); Lupardus et al., “A Requirement for Replication in Activation of the ATR-Dependent DNA Damage Checkpoint,” Genes Dev. 16:2327-2332 (2002)). In contrast, ATM is more important for the Ionizing Radiation (IR)-induced DNA damage checkpoint. Both proteins are targets for methylxanthines. ATR acts in concert with RepC-like protein Rad17 and the proliferating cell nuclear antigen (PCNA)-like heterotrimer of Rad9, Hus1, and Rad1 to enforce the DNA damage checkpoint (Bao et al., “ATR/ATM-Mediated Phosphorylation of Human Rad17 is Required for Genotoxic Stress Responses,” Nature 411:969-974 (2001); Roos-Mattjus et al., “Genotoxin-Induced Rad9-Hus1-Rad1(9-1-1) Chromatin Association is an Early Checkpoint Signaling Event,” J. Biol. Chem. 277:43809-43812 (2002); Zou et al., “Regulation of ATR Substrate Selection by Rad17-Dependent Loading of Rad9 Complexes onto Chromatin,” Genes Dev. 16:198-208 (2002)). The budding yeast Mec1 protein kinase is homologous to the mammalian ATR; Mec1 associates with proteins homologous to those that associate with ATR in mammalian cells to enforce the DNA damage checkpoint in the budding yeast (Melo et al., “Two Checkpoint Complexes are Independently Recruited to Sites of DNA Damage in vivo,” Genes Dev. 15:2809-2821 (2001)). Both Meel and ATR deletions are lethal in their respective organisms. However, the lethality associated with Mec1 deletion can be rescued by a deletion in the Sml1 gene, which controls nucleotide synthesis in yeast.

Several human viruses, including reovirus as well as human papillomavirus, encode gene products that are capable of induction of G2 phase cell cycle arrest. However, the induction of cell cycle arrest by the HIV-1 Vpr and the related Vpr gene products of primate lentiviruses has been most extensively studied. Infection with HIV-1 leads to the accumulation of infected cells in the G₂ phase of the cell cycle. This phenomenon can be explained by the action of a single HIV-1 encoded protein, Viral Protein R (Vpr), that is both necessary and sufficient for the cell cycle arrest (He et al., “Human Immunodeficiency Virus Type 1 Viral Protein R (Vpr) Arrests Cells in the G2 Phase of the Cell Cycle by Inhibiting p34cdc2 Activity,” J. Virol. 69:6705-6711 (1995); Jowett et al., “The Human Immunodeficiency Virus Type 1 vpr Gene Arrests Infected T Cells in the G2+M Phase of the Cell Cycle,” J. Virol. 69:6304-6313 (1995); Re et al., “Human Immunodeficiency Virus Type 1 Vpr Arrests the Cell Cycle in G2 by Inhibiting the Activation of p34cdc2-Cyclin B,” J. Virol. 69:6859-6864 (1995); Rogel et al., “The Human Immunodeficiency Virus Type 1 vpr Gene Prevents Cell Proliferation During Chronic Infection,” J. Virol. 69:882-888 (1995); Shostak et al., “Roles of p53 and Caspases in the Induction of Cell Cycle Arrest and Apoptosis by HIV-1 vpr,” Exp Cell Res. 251:156-165 (1999); Stewart et al., “Human Immunodeficiency Virus Type 1 Vpr Induces Apoptosis Following Cell Cycle Arrest,” J. Virol. 71:5579-5592 (1997)). The role of the cell cycle arrest in the HIV-1 pathogenesis is unclear. Vpr-induced G₂ arrest leads to moderate transactivation of the HIV-1 promoter, the long terminal repeat (LTR) (Forget et al., “Human Immunodeficiency Virus Type 1 vpr Protein Transactivation Function: Mechanism and Identification of Domains Involved,” J. Mol. Biol. 284:915-923 (1998); Goh et al., “HIV-1 Vpr Increases Viral Expression by Manipulation of the Cell Cycle: A Mechanism for Selection of Vpr in vivo,” Nat Med. 4:65-71 (1998); Hrimech et al., “Human Immunodeficiency Virus Type 1 (HIV-1) Vpr Functions as an Immediate-Early Protein During HIV-1 Infection,” J. Virol. 73:4101-4109 (1999); Zhu et al., “Comparison of Cell Cycle Arrest, Transactivation, and Apoptosis Induced by the Simian Immunodeficiency Virus SIVagm and Human Immunodeficiency Virus Type 1 vpr Genes,” J. Virol. 75:3791-3801 (2001)).

The G2 phase arrest and subsequent apoptosis may explain aspects of the CD4⁺ cell death. Vpr-induced cell cycle arrest has been extensively documented in a diverse array of eukaryotic cells, from the primary lymphocytes to transformed mammalian cell lines to yeast, suggesting an involvement of a highly conserved pathway. Despite extensive efforts to elucidate the cellular pathway in question, it has remained enigmatic. Early studies demonstrated that Vpr-induced G₂ arrest is associated with inactivation of the cyclin-dependent kinase Cdc2 by hyperphosphorylation and concomitant suppression of cdc2/cyclinB kinase activity that is necessary for the G2 to M transition. In response to Vpr, Cdc2-specific phosphatase, Cdc25C, is hyperphosphorylated in a pattern consistent with inactivation. These observations, coupled with sensitivity of the Vpr-induced G2 arrest to radiosensitizing agents, have led to the suggestion that Vpr induces cell cycle arrest via a DNA-damage sensitive pathway (He et al., “Human Immunodeficiency Virus Type 1 Viral Protein R (Vpr) Arrests Cells in the G2 Phase of the Cell Cycle by Inhibiting p34cdc2 Activity,” J. Virol. 69:6705-6711 (1995); Jowett et al., “The Human Immunodeficiency Virus Type 1 vpr Gene Arrests Infected T Cells in the G2 +M Phase of the Cell Cycle,” J. Virol. 69:6304-6313 (1995); Poon et al., “Human Immunodeficiency Virus Type 1 vpr Gene Induces Phenotypic Effects Similar to Those of the DNA Alkylating Agent, Nitrogen Mustard,” J. Virol. 71:3961-3971 (1997); Re et al., “Human Immunodeficiency Virus Type 1 Vpr Arrests the Cell Cycle in G2 by Inhibiting the Activation of p34cdc2-Cyclin B,” J. Virol. 69:6859-6864 (1995)). A direct binding of Vpr to DNA has been reported (Zhang et al., “Direct Binding to Nucleic Acids by Vpr of Human Immunodeficiency Virus Type 1,” Gene 212:157-166 (1998)), however the possibility that Vpr activates DNA damage-dependent cellular pathways by directly causing alterations in the structure or the integrity of DNA has not been demonstrated.

It has been shown that Vpr-induced G₂ arrest is independent of ATM function (Bartz et al., “Human Immunodeficiency Virus Type 1 Cell Cycle Control: Vpr is Cytostatic and Mediates G2 Accumulation by a Mechanism Which Differs From DNA Damage Checkpoint Control,” J. Virol. 70:2324-2331 (1996)). In addition, p53, which is associated with DNA-damage response, is not necessary for the vpr-mediated cell cycle arrest or apoptosis (Bartz et al., “Human Immunodeficiency Virus Type 1 Cell Cycle Control: Vpr is Cytostatic and Mediates G2 Accumulation by a Mechanism Which Differs From DNA Damage Checkpoint Control,” J. Virol. 70:2324-2331 (1996); Shostak et al., “Roles of p53 and Caspases in the Induction of Cell Cycle Arrest and Apoptosis by HIV-1 vpr,” Exp Cell Res. 251:156-165 (1999)). The role of ATR in the Vpr-induced cell cycle arrest has not previously been addressed. Therefore, it would be desirable to identify both the DNA damage-signaling pathway that is initiated by ATR and its signaling partners in vpr-induced G₂ arrest, as well as identify inhibitors of ATR and Rad17.

The present invention is directed to overcoming these and other deficiencies in the art.

### SUMMARY OF THE INVENTION

A first aspect of the present invention relates to a method of inhibiting HIV replication that includes: contacting a cell susceptible to HIV infection with an effective amount of an inhibitor of ATR or Rad17, or an inhibitor of an ATR-controlled pathway, under conditions effective to inhibit HIV replication in the cell.

A second aspect of the present invention relates to a method of treating or preventing an HIV infection that includes: administering to a patient an amount of an inhibitor of ATR or Rad17, or an inhibitor of an ATR-controlled pathway, which is effective to inhibit HIV replication in a cell susceptible to HIV infection.

A third aspect of the present invention relates to a method of inhibiting the activity of a viral protein R (Vpr) that includes: contacting ATR or Rad17 with an effective amount of an inhibitor of ATR or Rad17, respectively, or contacting a component of an ATR-controlled pathway with an inhibitor thereof, under conditions effective to inhibit viral protein R activity which occurs via a pathway under regulatory control of ATR or Rad17.

A fourth aspect of the present invention relates to a method of treating a latent HIV infection in a patient that includes: administering to a patient a first agent that activates latently infected patient cells to induce HIV Vpr expression; and administering to the patient a second agent that activates an ATR-controlled pathway.

A fifth aspect of the present invention relates to an inhibitory RNA molecule that binds to mRNA encoding ATR under conditions effective to inhibit expression of the mRNA.

A sixth aspect of the present invention relates to a DNA molecule encoding the inhibitory RNA molecule of the present invention, as well as DNA constructs containing the DNA molecule, expression vectors containing the DNA construct, and host cells transformed with the DNA construct.

Vpr-induced G2 arrest has deleterious effects on HIV-1 infected cells and on immune function in general. Generation of the immune response depends on activation by antigen and subsequent proliferation of helper T cells. Activated T-lymphocytes infected with HIV-1 are unable to undergo clonal proliferation due to Vpr-induced G2 arrest. Without being bound by belief, it is expected that limiting this critical phase of the cellular immune response is likely to disrupt downstream events, including T cell helper activity via release of cytokines, stimulation, and modulation of the humoral immune responses, generation of memory cells, and elicitation of cytotoxic T cell activity. Thus, it is likely that vpr may constitute a major determinant of cell death in vivo as well. Targeting of the Vpr protein or its mode of action in accordance with the present invention offers new avenues for therapeutic intervention in the pathogenesis of AIDS. In particular, it is believed that the blocking of vpr action will reduce the efficiency of viral replication by 5- to 20-fold per replication cycle.

### BRIEF DESCRIPTION OF THE DRAWINGS

FIG. 1A illustrates viral vectors used for transduction of HIV-1 Vpr, and murine HSA. Only the viral open reading frames (ORFs) are shown. Both vectors contain all the HIV ORFs with the following exceptions: env and nef were deleted in both DHIV-VPR and DHIV-HAS. The green fluorescent protein (GFP) ORF was inserted in replacement of nef to allow detection of the infected cells; in DHIV-HSA, vpr was replaced with HSA. FIG. 1B illustrates additional viral vectors for transduction of HIV-1 Vpr and GFP. pHA-GFP is a null vector which allows for detection of infected cells by expression of the GFP. pHA-VPR-IRES-GFP (hereinafter designated pHA-VPR) encodes Vpr and GFP, and includes an internal ribosome entry site (IRES). GFP is again used to detect transfected cells.

FIG. 2 is an image of a Western blot which demonstrates that the DHIV-VPR vector, expressing Vpr, induces phosphorylation of Cdc2 at Tyr15 in infected HeLa cells at forty-eight hours post-infection, while the DHIV-HAS vector, expressing mHSA, does not HeLa cells expressing Vpr (lane 2) had increased levels of Cdc2 Tyr15 phosphorylation when compared to either mock-infected (lane 1) or mHSA-expressing cells (lane 3) cells. The effects of caffeine (lane 4), taxol (lane 5), and doxorubicin (lane 6) are also shown.

FIGS. 3A-B illustrate the flow cytometric analysis of Vpr-induced G2 arrest. In FIG. 3A, HeLa cells were treated with either DMSO, LY294002 (an inhibitor of PI3K (Smith et al., “DNA-dependent Protein Kinase and Related Proteins,” Biochem. Soc. Symp. 64:91-104 (1999); Vlahos et al., “A Specific Inhibitor of Phosphatidylinositol 3-kinase, 2-(4-morpholinyl)-8-phenyl-4H-1-benzopyran-4-one (LY294002),” J. Biol. Chem. 269:5241-5248 (1994), each of which is hereby incorporated by reference in its entirety)), or caffeine, and then infected with either DHIV-VPR or DHIV-HSA. At 24 hours post infection, cell cycle profiles were analyzed by measuring the DNA content. Histograms depict the cell cycle profiles. The left peaks constitute cells in G1, and the right peaks constitute cells in G2/M; cells in S phase are between the G1and G2 peaks. In FIG. 3B, GM847ATRkd cells were either induced to express ATRkd by addition of 2mM doxycycline forty-eight hours prior to infection, or left untreated. ATRkd is a kinase-deficient ATR that carries an Asp(2475) to Ala substitution within its catalytic domain (Cliby et al., “Overexpression of a Kinase-inactive ATR Protein Causes Sensitivity to DNA-damaging Agents and Defects in Cell Cycle Checkpoints,” EMBO J. 17:159-169 (1998), which is hereby incorporated by reference in its entirety), is defective in autophosphorylation, and when expressed in mammalian cells acts as a dominant-negative regulator of wild type ATR. GM847/ATRkd is a human fibroblast cell line that was stably transduced with a tetracycline inducible version of ATRkd (Cliby et al., “Overexpression of a Kinase-inactive ATR Protein Causes Sensitivity to DNA-damaging Agents and Defects in Cell Cycle Checkpoints,” EMBO J. 17:159-169 (1998), which is hereby incorporated by reference in its entirety). The frequencies of cells in different stages of the cell cycle were calculated using Multicycle AV software (Phoenix Flow Systems, San Diego, Calif.).

FIG. 4 is an illustration of a construct designed for expression of siRNAs from an RNA polymerase III-specific promoter.

FIG. 5 illustrates cell cycle analyses obtained following transfection of HeLa cells with pHR-VPR (VPR+) that have been transfected with ATR specific-RNAi, designated (+) RNAi, or an empty vector, designated (−) RNAi. Transduction of Vpr in the ATR specific-RNAi transfected cells (VPR(+)/(+)RNAi) yielded a significantly attenuated G2 arrest as compared with HeLa cells transfected with empty vector (VPR(+)/(−)RNAi).

FIG. 6 is an image of a Western blot examining the phosphorylation status of Chk1 in HeLa cells that were mock infected (lane 1), infected with pHR-VPR (lane 2), infected with pHR-GFP (lane 3), or treated with doxycyclin (lane 4). All cells displayed a similar level of Chk1 expression (lower panel), but only the cells infected with DHIV-VPR or exposed to doxycyclin displayed an additional slower migrating band corresponding to phosphorylated Chk1.

FIG. 7 are cell cycle analyses of HeLa cells incubated with either 200 nM UCN-01 (middle column) or 2 nM caffeine (right column) in conjunction with incubation with 1 μM Etoposide (first row), pHR-GFP transfection (second row), pHR-VPR transfection (third row), incubation with 1 μM Doxorubicin (fourth row), or incubation with 25 nM Taxol (fifth row). Incubation with UCN-01 resulted in dramatic reduction of Vpr-induced G2 arrest, consistent with observations that UCN-01 reduced doxorubicin-induced G2 arrest.

FIG. 8 illustrates cell cycle analyses obtained following transfection of HeLa cells with pHR-VPR (VPR+) that have been transfected with Rad17 specific-RNAi, designated (+) RNAi, or an empty vector, designated (−) RNAi. Transduction of Vpr in the Rad17 specific-RNAi transfected cells (VPR(+)/(+)RNAi) yielded a significantly attenuated G2 arrest as compared with HeLa cells transfected with empty vector (VPR(+)/(−)RNAi).

FIG. 9 is an image of Saccharomyces cerevisiae cells transformed with and empty vector or a Vpr-encoding vector under a methionine inducible promoter, P-MLT:Vpr. Vpr effectively caused G2 arrest in the budding yeast, as evidence by “dumbbell” shaped cells.

### DETAILED DESCRIPTION OF THE INVENTION

The present invention relates to methods of inhibiting HIV replication in cells, inhibiting the activity of Vpr which occurs via a pathway under regulatory control of ATR and Rad17, and treating or preventing an HIV infection, all of which involve the use of inhibitors of ATR or Rad17. A further aspect of the present invention relates to a method of identifying inhibitors of ATR or Rad17.

The present invention also relates to novel inhibitors of ATR or Rad17, more specifically inhibitory RNA molecules that bind to mRNA encoding ATR or Rad17, and DNA molecules encoding the same.

One aspect of the present invention relates to a method of inhibiting the activity of an HIV Vpr that includes contacting ATR or Rad17 with an effective amount of an inhibitor of ATR or Rad17, respectively, under conditions effective to inhibit Vpr activity which occurs via a pathway under regulatory control of ATR or Rad17. In accordance with this aspect of the present invention, the Vpr can be present in vitro or in vivo, as described hereinafter.

As used herein, “Vpr” refers to any lentivirus Vpr protein, but preferably an HIV-1 Vpr protein as described by Cohen et al., “Human Immunodeficiency Virus vpr Product is a Virion-associated Regulatory Protein,” J. Virol. (1990) 64:3097-3099 (1990), which is hereby incorporated by reference in its entirety, or an HIV-2 Vpr protein as described by Dedera et al., “Viral Protein R of Human Immunodeficiency Virus Types 1 and 2 is Dispensable for Replication and Cytopathogenicity in Lymphoid Cells,” J. Virol. 63(7):3205-3208 (1989), which is hereby incorporated by reference in its entirety), as well as the corresponding Vpr proteins derived from other lentiviruses. Vpr protein also includes minimally modified forms of this protein or subunits thereof which retain the ability to arrest the cell cycle at the G2 stage. It is well understood that minor modification can be made to the amino acid sequence of proteins without altering, dramatically, their activity. Preferred modifications include substitution of conservative amino acids for those in the wild-type protein in noncritical regions. Minimal numbers of substitutions are preferred. In addition, it is also understood that the primary amino acid structure may be derivatized to, for example, sugars, lipids, acyl groups, and the like. Such modifications which do not interfere with the G2- arresting function of Vpr are also contemplated. Furthermore, the complete amino acid sequence may not be necessary for the requisite activity. Thus, fragments of the wild-type Vpr which remain active are also included.

In general, “Vpr” as used in the present context, includes any altered forms of wild-type Vpr proteins which remain useful in the method of the invention. The test for ascertaining such utility is straightforward; the modified form need only be tested in comparison to wild type for its ability to arrest cells at the G2 stage or, more simply, to inhibit growth when expressed in mammalian or other eukaryotic cells (see, e.g., Planelles et al., “Vpr-induced Cell Cycle Arrest is Conserved Among Primate Lentivinises,” J. Virol. 70(4) 2516-2524 (1996), which is hereby incorporated by reference in its entirety).

As used herein, ATR refers to the ATM and Rad3 -related protein as described in Genbank Accession NM_{—}001184 (and references cited therein) and Bentley et al., “The Schizosaccharomyces pombe rad3 Checkpoint Gene,” EMBO J. 15:6641-6651 (1996), each of which is hereby incorporated by reference in its entirety, as well as naturally occurring variants thereof (i.e., wild-type), and minimally modified forms of the protein which retain their kinase activity with respect to Cik1. ATR is preferably human ATR, although homologs from other eukaryotes is also encompassed.

As used herein, Rad17 refers to the G2 cell cycle checkpoint protein as described in Genbank Accession NM_{—}133338 (and references cited therein), which is hereby incorporated by reference in its entirety, as well as naturally occurring variants thereof (i.e., wild-type), and minimally modified forms of the protein which retain their ability to regulate ATR substrate selection. Rad17 is a RepC-like protein that is required for the ATR induced checkpoint activation (Bao et al., “ATR/ATM-Mediated Phosphorylation of Human Rad17 is Required for Genotoxic Stress Responses,” Nature 411:969-974 (2001), which is hereby incorporated by reference in its entirety) and regulates ATR substrate selection (Zou et al., “Regulation of ATR Substrate Selection by Rad17-Dependent Loading of Rad9 Complexes onto Chromatin,” Genes Dev. 16:198-208 (2002), which is hereby incorporated by reference in its entirety). Rad17 is preferably human Rad17, although homologs from other eukaryotes is also encompassed.

It is well understood that minor modification can be made to the amino acid sequence of ATR and Rad17 proteins without altering, dramatically, their activity. Preferred modifications include substitution of conservative amino acids for those in the wild-type protein in noncritical regions. Minimal numbers of substitutions are preferred. In addition, it is also understood that the primary amino acid structure may be derivatized to, for example, sugars, lipids, acyl groups, and the like. Such modifications which do not interfere with the ATR kinase activity on Chk1 or the Rad17 regulated substrate selection for ATR are contemplated. Furthermore, the complete amino acid sequence may not be necessary for the requisite activity. Thus, fragments of the wild-type ATR or Rad17 which remain active are also included.

In general, ATR or Rad17 as used herein includes any altered forms of wild-type ATP or Rad17 proteins, respectively, which remain useful in the methods of the invention. The test for ascertaining such utility is straightforward: for ATR, the modified form need only be tested in comparison to wild type for its ability to phosphorylate Chk1 ; for Rad17, the modified need only be tested in comparison to wild type for its ability to regulate ATR substrate selection (i.e., by loading Rad9 complexes onto chromatin).

Without being bound by belief, it is believed (as demonstrated infra) that that ATR or Rad17 inhibition has the ability to interfere with Vpr-induced G2 cell cycle arrest. By interfering with Vpr-induced G2 cell cycle arrest, it is possible to allow arrested cells to continue toward the M phase of the cell cycle, thereby preventing virus particle replication in HIV infected cells and consequently inhibiting HIV infectivity within the cells, as well as providing a treatment for HIV infection in a patient or preventing development of an HIV infection.

A further aspect of the present invention therefore relates to a method of inhibiting HIV replication that includes contacting a cell susceptible to HIV infection with an effective amount of an inhibitor of ATR or Rad17 under conditions effective to inhibit HIV replication in the cell. The contacting is carried out under conditions effective to inhibit G2 cell cycle arrest that is normally induced by HIV infection of the cell (specifically Vpr), wherein inhibition of G2 cell cycle arrest allows cell cycle progression to occur, thereby inhibiting HIV replication and, hence, infectivity.

Cells in which ATR or Rad17 activity is to be disrupted include any cell that is susceptible to HIV infection, which is generally regarded as those cells that possess the CD4 cell surface marker (CD4⁺ cells). CD4⁺ cells can include, without limitation, T cells, macrophage, and other lymphoid and non-lymphoid cells. Of these, ATR and Rad17 disruption preferably occurs in CD4⁺ T cells and/or macrophage. The cells in which ATR or Rad17 activity is to be disrupted can be located in vivo (i.e., within an organism) or ex vivo.

In accordance with the present invention, inhibitors of ATR or Rad17 can either inhibit ATR or Rad17 activity directly, by binding to ATR or Rad17 or their substrates to interfere with ATR activity or Rad17 as described above, or by interfering with members of an ATR-controlled pathway, such as the ATR→Chk1→Cdc25→Cdc2 pathway that regulates G2 to M phase transition; or by inhibiting the production of ATR or Rad17 via interference with the expression of these proteins (i.e., interfering with translation or transcription processes).

The inhibitors of ATR or Rad17 can be small molecules, peptides or polypeptides, or nucleic acid molecules.

Exemplary inhibitors of ATR include, without limitation, caffeine and 2-(4-morpholinyl)-8-phenyl-4H-1-benzopyran-4-one (LY294002). An inhibitor of one member of the ATR-controlled pathway is 7-hydroxystaurosporine (UCN01), which is an inhibitor of Chk1. Suitable inhibitors of ATR expression include, without limitation, inhibitory RNA molecules, preferably those that are less than about 30 nucleotides in length, more preferably about 19-23 nucleotides in length. The inhibitory RNA molecules that interfere with ATR expression are short interfering RNA molecules (siRNAs) that target (or bind to) an ATR MnRNA sequence.

Two exemplary inhibitory RNA molecules targeted to ATR mRNA sequences are characterized by the following sense-strand nucleotide sequences:

(SEQ ID NO:1)

CCUCGGUGAUGUUGCUUGAUU
and

(SEQ ID NO:2)

GCCAUGAGCGCAAAGGCAGUU

Other ATR inhibitors can be identified at the Ambion, Inc. Internet site, which provides a target sequence to siRNA converter, identifying the sense and anti-sense strands of the siRNA molecule, as well as identifying the DNA construct needed to express the siRNA.

Suitable inhibitors of Rad17 expression include, without limitation, inhibitory RNA molecules, preferably those that are less than about 30 nucleotides in length, more preferably about 19-23 nucleotides in length. The inhibitory RNA molecules that interfere with Rad17 expression are short interfering RNA molecules (siRNAs) that target (or bind to) a Rad17 mRNA sequence.

An exemplary inhibitory RNA molecules targeted to Rad17 mRNA sequences is characterized by the following sense-strand nucleotide sequence:

(SEQ ID NO:3)

CAGACUGGGUUGACCCAUCUU

Inhibitory RNA molecules targeted to Rad17 mRNA are also described in Zou et al., “Regulation of ATR Substrate Selection by Rad17-Dependent Loading of Rad9 Complexes onto Chromatin,” Genes Dev. 16:198-208 (2002), which is hereby incorporated by reference in its entirety). Other Rad17 inhibitors can be identified at the Ambion, Inc. Internet site, which provides a target sequence to siRNA converter, identifying the sense and anti-sense strands of the siRNA molecule, as well as identifying the DNA construct needed to express the siRNA.

Inhibitory RNA molecules of the present invention can be produced intracellularly using recombinant procedures, as described in Brummelkamp et al., “A System for Stable Expression of Short Interfering RNAs in Mammalian Cells,” Science 21:21 (2002), which is hereby incorporated by reference in its entirety. Basically, intracellular transcription of siRNAs can be achieved by cloning the siRNA templates into RNA pol III transcription units, which normally encode the smaller nuclear RNA U6 or the human RNAse P RNA H1. Two approaches have been developed for expressing siRNA: in the first, sense and antisense strands constituting the siRNA duplex are transcribed by individual promoters; in the second (see FIG. 4), siRNAs are expressed as foldback stem-loop structures that are processed into the siRNAs. The U6 and H1 promoters are members of the type III class of Pol III promoters. U6 and H1 are different in size but contain the similar conserved sequence elements or protein binding sites. The +1 nucleotide of the U6-like promoters is always guanosine, whereas the +1 for H1 promoters is adenosine. The termination signal for these promoters is defined by 5 thymidines, and the transcript is typically cleaved after the second uridine. Cleavage at this position generates a 3′ UU overhang in the expressed siRNA, which is identical to the 3′ overhangs of synthetic siRNAs. Any sequence less than about 400 nucleotides in length can be transcribed by these promoters, therefore they are ideally suited for the expression of short siRNAs of approximately 50-nucleotide RNA stem-loops.

Because the inhibitory RNA molecules are produced intracellulary, it is intended that DNA molecules encoding the inhibitory RNA molecule can be administered or taken up by cells, of the type described above, in which ATR or Rad17 activity is to be disrupted.

The DNA molecule is preferably a DNA construct of the type described above and illustrated in FIG. 4, which includes a DNA molecule encoding the RNA molecule, operably coupled at the 5′ end thereof to a promoter-effective DNA molecule and operably coupled at the 3′ end thereof to a transcription termination signal. As shown in FIG. 4, an H1 promoter and a five thymidine transcription termination signal are utilized in a preferred embodiment of the invention.

The DNA molecule can be introduced into cells located in vivo (for inhibition of ATR or Rad17 therein) or ex vivo (for either inhibition of ATR or Rad17 therein, or for recombinant production of inhibitory RNAs of the present invention).

Ex vivo uptake by cells can be achieved via transfection, transduction, mobilization, electroporation, or calcium phosphate precipitation. Of these approaches, electroporation and calcium phosphate precipitation are preferred. For transfection, any number of suitable transfection media can be used to enhance ex vivo transformation of particular cell types, including without limitation, Polyfect® (Westburg), jetSI™ (Q-BIOgene), TransMessenger (Qiagen), and ExGen 500 (Fermentas).

Suitable host cells include, but are not limited to, bacteria, yeast, mammalian cells, and the like. Exemplary mammalian cells include any of the above-identified CD4⁺ cells as well as cell lines such as, among others, COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, and NS-1 cells.

For ex vivo transformation and subsequent recombinant expression and isolation of the inhibitory RNAs of the present invention, the cells are preferably mammalian cell lines of the type described above. The inhibitory RNAs can be isolated and purified using standard nucleic acid isolation techniques. Once isolated, they can then be introduced into CD4⁺ cells via administration to a patient in accordance with the present invention (described hereinafter).

In vivo uptake by cells, i.e., CD4⁺ cells, can be achieved using any of a variety of recombinant expression vectors including, without limitation, adenoviral vectors, retroviral vectors, and lentiviral vectors.

Adenovinis gene delivery vehicles can be readily prepared and utilized given the above-identified procedures for preparation of DNA constructs encoding siRNAs and the disclosure provided in Berkner, Biotechniques 6:616-627 (1988) and Rosenfeld et al., Science 252:431-434 (1991), WO 93/07283, WO 93/06223, and WO 93/07282, each of which is hereby incorporated by reference in its entirety. In vivo use of adenoviral vehicles is described in Flotte et al., Proc. Nat'l Acad. Sci. 90:10613-10617 (1993); and Kaplitt et al., Nature Genet. 8:148-153 (1994), each of which is hereby incorporated by reference in its entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; and U.S. Pat. No. 5,885,808 to Spooner et al.; and U.S. Pat. No. 5,871,727 to Curiel, each of which is hereby incorporated by reference in its entirety).

Retroviral vectors which have been modified to form infective transformation systems can also be used to deliver DNA constructs of the present invention into a target cell. One such type of retroviral vector is disclosed in U.S. Pat. No. 5,849,586 to Kriegler et al., which is hereby incorporated by reference in its entirety.

Lentiviral gene delivery vehicles can be readily prepared and utilized given the above-identified procedures for preparation of DNA constructs encoding siRNAs and the disclosure provided in U.S. Pat. No. 6,498,033 to Dropulic et al., U.S. Pat. No. 6,428,953 to Naldini et al., U.S. Pat. No. 6,277,633 to Olsen, U.S. Pat. No. 6,235,522 to Kingsman, U.S. Pat. No. 6,207,455 to Chang, and U.S. Pat. No. 6,165,782 to Naldini et al., each of which is hereby incorporated by reference in its entirety. Basically, lentiviral gene delivery vehicles are desirable, because HIV-derived vectors can be prepared to target the CD4⁺ cells in accordance with the present invention.

A still further aspect of the present invention relates to a method of treating or preventing an HIV infection that includes administering to a patient an amount of an inhibitor of ATR or Rad17 which is effective to inhibit HIV replication in a cell susceptible to HIV infection. The administration of ATR or Rad17 inhibitors in accordance with this aspect of the present invention is effective to inhibit G2 cell cycle arrest in the cells of the patient (i.e., following HIV infection of the cell), wherein inhibition of G2 cell cycle arrest allows cell cycle progression to occur, thereby inhibiting HIV replication and, hence, infectivity.

By administering such inhibitors of ATR or Rad17 prior to HIV infection in the patient, it is possible to prevent HIV infection from developing because CD4⁺ cells that are initially infected by the HIV will likely avoid G2 cell cycle arrest, thereby allowing the patient's immune response to defeat the initial infection. The expected mechanism leading to delayed ability to replicate would be a direct consequence of the decreased transcriptional activity of the viral promoter, the LTR, under conditions where ATR is inhibited. Although the inhibition per replication cycle would be expected to be between 5- and 20-fold, when compounded over many replication cycles, it is expected to have dramatic negative effects on virus replication and perhaps maintenance.

By administering such inhibitors of ATR or Rad17 after HIV infection in the patient, it is possible to treat the infection in a manner which either reduces the patient's viral load or otherwise prevents an increase in the patient's viral load at a rate that would otherwise occur in the absence of any treatment. As a result, it is possible to treat such patients in a manner which either prevents or delays the onset of AIDS. It may also have synergistic or additive effects when-used in combination with antiretroviral therapy (i.e., drug cocktail).

As an alternative to recombinant techniques for inhibiting ATR or Rad17 in CD4⁺ cells in vivo (described above), the inhibitors of ATR or Rad17 can be administered to a patient under conditions effective to cause uptake of the ATR or Rad17 inhibitor by those cells. Unlike cells transfected with an siRNA expression vector as described above, which would experience steady, long-term mRNA inhibition, cells transfected with exogenous synthetic siRNAs typically recover from mRNA suppression within seven days or ten rounds of cell division. Likewise, cells into which other inhibitors of ATR or Rad17 have been introduced will similarly be expected to experience transient ATR or Rad17 inhibition. Nonetheless, such inhibitors of ATR or Rad17 can be utilized to provide short term inhibition to HIV infection, although with repeated administration continued inhibition to HIV infection can be achieved.

Delivery of inhibitors of ATR or Rad17 can be achieved by use of liposomes or other suitable delivery vehicles. Basically, this involves providing a liposome which includes the inhibitor to be delivered, and then contacting the CD4⁺ cell with the liposome under conditions effective for delivery of the inhibitor into the cell.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), each of which is hereby incorporated by reference in their entirety). When liposomes are endocytosed by a target CD4⁺ cell, for example, they can be routed to acidic endosomes which will destabilize the liposome and result in drug release.

Alternatively, the liposome membrane can be chemically modified such that an enzyme is placed as a coating on the membrane which slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

This liposome delivery system can also be made to accumulate at the targeted CD4⁺ cells via active targeting (e.g., by incorporating an antibody or hormone on the surface of the liposomal vehicle). This can be achieved according to known methods.

Different types of liposomes can be prepared according to Bangham et al., J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., each of which is hereby incorporated by reference in their entirety.

Whether the inhibitors of ATR or Rad17 are administered alone or in combination (as a pharmaceutical composition) with pharmaceutically or physiologically acceptable carriers, excipients, or stabilizers, or in solid or liquid form such as, tablets, capsules, powders, solutions, suspensions, or emulsions, they can be administered orally, parenterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes, or by transdermal delivery. For most therapeutic purposes, the inhibitors of ATR or Rad17 can be administered intravenously.

For injectable dosages, solutions or suspensions of these materials can be prepared in a physiologically acceptable diluent with a pharmaceutical carrier. Such carriers include sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

For use as aerosols, the inhibitors of ATR or Rad17 in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The inhibitors of ATR or Rad17 can also be administered from extended release formulations that can be implanted in a patient. Polymeric delivery vehicles for sustained release of active agents are well known in the art and can be optimized for delivery of the inhibitors of ATR or Rad17 in accordance with the present invention.

Compositions within the scope of this invention include all compositions wherein the inhibitor of ATR or Rad17 is contained in an amount effective to achieve its intended purpose. While individual needs vary, determination of optimal ranges of effective amounts of each component is within the skill of the art. Typical dosages comprise about 0.01 to about 100 mg/kg·body wt. The preferred dosages comprise about 0.1 to about 100 mg/kg·body wt. The most preferred dosages comprise about 1 to about 100 mg/kg·body wt. Treatment regimen for the administration of the compounds of the present invention can also be determined readily by those with ordinary skill in art.

In addition to the foregoing treatments, applicant's identification of ATR and Rad17 as being implicated in the G2 cell cycle arrest caused by HIV Vpr expression in infected cells affords an assay for developing still farther inhibitors of ATR and Rad17. The assay includes treating a cell that contains Vpr and is in G2 cell cycle arrest with a putative inhibitor of ATR or Rad17, and then determining whether the cell remains in the G2 phase (indicating that the putative inhibitor of ATR or Rad17 is ineffective) or whether the cell progresses from the G2 phase (indicating ATR or Rad17 inhibition). Typically, the assay is performed on a population of cells, whereby a significant change in the population size that remains in G2 is indicative of such inhibition.

As an alternative approach for treating an existing HIV infection, rather than inhibiting the ATR or Rad17 regulated inhibition of the G2 to M transition, the ATR-controlled pathway is instead activated early in the infection process to force the cell into G2 arrest and, ultimately programmed cell death. When patients undergo triple drug therapy, in most cases the virus becomes latent and forms a long-lived reservoir in the body. There is great interest in developing methods to eliminate this reservoir, so that therapy can be discontinued without the virus becoming activated and taking over again. One approach is to use a cytokine, such as IL-2, or a mitogenic stimulus to activate latently infected cells and “flush out” latent virus. Of course, the downside of that is that new virus is produced and, although the patient is under therapy and in theory the virions would be inactive, certain virions may actually escape the effects of the therapy and re-establish latency in other cells. This aspect of the present invention provides for the use of HIV's own ability to kill the host cell (the previously quiescent cell that is now in an activated state) to prevent production of progeny. Activation of a latent provirus would involve activation of viral gene expression, including expression of Vpr. To potentiate the cytostatic and cytotoxic effects of Vpr, one or more activators of the ATR-controlled pathway are administered to the patient. Because the ATR-controlled pathway is activated by a variety of genotoxic agents, any such genotoxic agents can be employed with activation of latent HIV. Suitable genotoxic agents are generally cancer therapeutics that induce DNA damage, such as (without limitation) doxorubicin, etoposide, radiation, etc. Without being bound by belief, the activation of ATR by two independent agents (HIV Vpr plus the genotoxic agent) will result in synergistic or additive effects, and potent activation of ATR will lead to rapid cell death. It is also possible that the use of a genotoxic drug or radiation may, by itself, act as an activation stimulus, bringing HIV out of latency. The administration of the agent that activates latently infected cells and the administration of the agent that activates the ATR-controlled pathway can occur simultaneously, or either one prior to the other.

### EXAMPLES

The following Examples are intended to be illustrative and in no way are intended to limit the scope of the present invention.

Materials and Methods

Cell lines: Human cervical cancer cell line HeLa and transformed human embryonic kidney cell line HEK293T were grown in DMEM 10% FBS. Human SV40 transformed fibroblasts GM847/ATRkd (a generous gift of Dr. Cimprich (Stanford) and Dr. Handeli (University of Washington)) and human osteosarcoma-derived U2OS ATRkd cell lines were maintained in DMEM 10% FBS with 400 μg/ml G418 and 200 μg/ml Hygromycin.

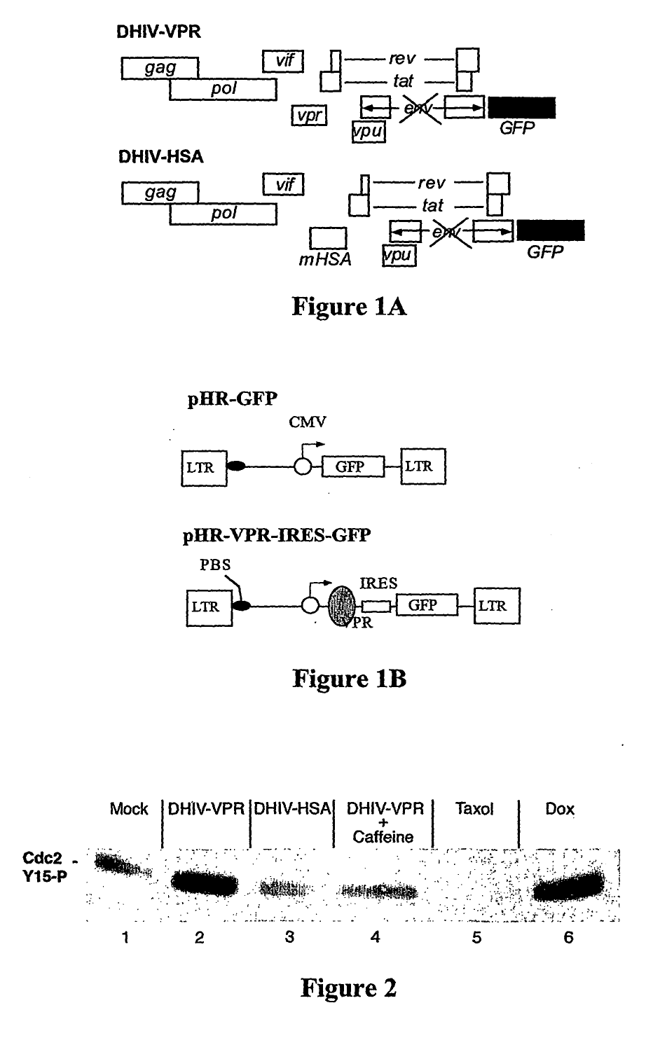

Viral vector production and titration: Lentiviral vectors were produced by transient transfection of 293T cells. For defective lentivirus vector production, pHR-GFP or pHR-VPR plasmids (FIG. 1B), were cotransfected with pCMVΔ8.2ΔVpr (An et al., “An Inducible Human Immunodeficiency Virus Type 1 (HI-1) Vector Which Effectively Suppresses HIV-1 Replication,” J. Virol. 73:7671-7677 (1999), which is hereby incorporated by reference in its entirety) and HCMV-VSVG (Akkina et al., “High-efficiency Gene Transfer into CD34+ Cells with a Human Immunodeficiency Virus Type 1-Based Retroviral Vector Pseudotyped with Vesicular Stomatitis Virus Envelope Glycoprotein,” Gen. J. Virol. 70:2581-2585 (1996), which is hereby incorporated by reference in its entirety) using the calcium phosphate-mediated transfection. DHIV-VPR and DHIV-HAS (FIG. 1A) were similarly prepared. Virus supernatant was collected at 48, 72 and 96 hours post-transfection. Harvested supernatants were cleared by low-speed centrifugation at 2,000 rpm and then frozen at −80° C. Vector titers were measured by infection of HeLa cells as described below, followed by flow cytometric analysis of cells positive for the reporter molecule, GFP. Vector titers were calculated as follows:
Titer=[F×C₀/V]×D
where F=frequency of GFP (+) cells by flow cytometry; C₀=total number of target cells at the time of infection; V=volume of inoculum; and D=virus dilution factor. Virus dilution factor used for titrations was D=100. Total number of target cells at the time of infection was 10⁶.
siRNA Vector Production and Transfection: siRNA production was carried out in accordance with the procedures described by Brummelkamp et al. (“A System for Stable Expression of Short Interfering RNAs in Mammalian Cells,” Science 21:21 (2002), which is hereby incorporated by reference in its entirety), except with the insertion therein of nucleic acid coding for the ATR siRNA of SEQ ID NOs: 1 or 2, or Rad17 siRNA of SEQ ID NO: 3. Transfection was carried out by electroporation or lipofectamine.
Cell cycle analysis: Cells were infected with either pHR-VPR or pHR-GFP at a multiplicity of infection (MOI) of 2.5. Where greater than 90% infection rate was achieved, as measured by counting cells GFP positive cells, the cells were detached with 2mM EDTA, washed in phosphate-buffered saline (PBS), fixed with 70% ethanol for over 18 hours at 4° C., and then stained with propidium iodide solution (20 μg/ml propidium iodide, 11.25 kunitz units/ml RNase A, in PBS). Where less than 90% infection was achieved, the cells were fixed in 0.25% p-formaldehyde to preserve GFP fluorescence and only the GFP positive cells were gated by flow cytometry to represent the infected fraction of the cells. Flow cytometric analysis was performed in an Epics Elite ESP (Coulter Corp., Hialeah, Fla.). Cell cycle analysis was performed using Multicycle AV software (Phoenix Flow Systems, San Diego, Calif.). All cell cycle experiments were performed at least three times and typical results are shown.
Drug treatments: LY294002 (Cell Signaling) was used at 50 μM, Caffeine (Sigma, St Louis, Mo.) was used at 2.5 mM, Doxorubicin was used at 1 μM, and Taxol was used at 25 nM, etoposide was used at 1 μM. UCN01 (7-hydroxystaurosporine, NSC 638850) was obtained from NCI.
Western blot: For the CHK-1 and Cdc2 blots, HeLa cells were washed in PBS and lysed in modified RIPA buffer (Cell Signaling Research, Beverly, Mass.). 100 μg of protein was loaded onto a 10% SDS-PAGE gel and electrophoretically transferred to a PVDF membrane. The membranes were blocked in Tris-buffered saline, 0.2% Tween 20, and 5% nonfat dry milk, and probed with anti Cdc2Y15 polyclonal antibodies (1:1000 dilution; Cell Signaling Research), or with monoclonal antibodies directed against Chk1 (1:250 dilution; Santa Cruz, Santa Cruz, Calif.) or Cdc2 (Santa Cruz) followed by a horseradish peroxidase-linked anti-rabbit or anti-mouse secondary antibody (1:1000 dilution; Amersham, Arlington Heights, Ill.). Proteins were detected with the use of an enhanced chemiluminescence reagent (Pierce) and visualized with the use of Biomax film (Eastman Kodak, Rochester, N.Y.). All Western blots were repeated at least twice and results of a typical experiment are shown.

### Example 1

### Vpr Arrests Cell Prior to Mitosis Entry and Induces Hyperphosphorylation of Cdc2 on Tyr15

Vpr-arrested cells have tetraploid amount of DNA that is characteristic of either G₂ or M phase arrest. G₂ arrest is characterized by inactive Cdc2/Cyclin B complex, which is normally responsible for G2 to M transition. In contrast, mitotic arrest is characterized by maintenance of high cyclin B/cdc2 associated activity. In response to DNA damage, Cdc2/CyclinB inactivation and subsequent arrest is due to the inhibitory phosphorylation of Cdc2 on Tyr15. Upon entry to mitosis, Cdc2 is uniformly dephosphorylated. The hyperphosphorylated form of Cdc2 exhibits slower migration on SDS-PAGE than its counterpart, the active form (Draetta et al., “Activation of cdc2 Protein Kinase During Mitosis in Human Cells: Cell Cycle-Dependent Phosphorylation and Subunit Rearrangement,” Cell 54:17-26 (1988), which is hereby incorporated by reference in its entirety). Vpr expression leads to inactivation of the Cdc2/CyclinB complex, the appearance of the slower migrating Cdc2 band, and the reduction of Cdc2/Cyclin B1 kinase activity (Bartz et al., “Human Immunodeficiency Virus Type 1 Cell Cycle Control: Vpr is Cytostatic and Mediates G2 Accumulation by a Mechanism Which Differs From DNA Damage Checkpoint Control,” J. Virol. 70:2324-2331 (1996); Di Marzio et al., “Mutational Analysis of Cell Cycle Arrest, Nuclear Localization and Virion Packaging of Human Immunodeficiency Virus Type 1 Vpr,” J. Virol. 69:7909-7916 (1995); He et al., “Human Immunodeficiency Virus Type 1 Viral Protein R (Vpr) Arrests Cells in the G2 Phase of the Cell Cycle by Inhibiting p34cdc2 Activity,” J. Virol. 69:6705-6711 (1995); Poon et al., “Human Immunodeficiency Virus Type 1 vpr Gene Induces Phenotypic Effects Similar to Those of the DNA Alkylating Agent, Nitrogen Mustard,” J. Virol. 71:3961-3971 (1997); Re et al., “Human Immunodeficiency Virus Type 1 Vpr Arrests the Cell Cycle in G2 by Inhibiting the Activation of p34cdc2-Cyclin B,” J. Virol. 69:6859-6864 (1995), each of which is hereby incorporated by reference in its entirety).

In order to specifically test whether Vpr induces Cdc2 phosphorylation on Tyr-15, an antibody was used that recognizes Cdc2 only when it is phosphorylated on the Tyr-15. HeLa cells were infected with isogenic, defective HIV-1 viruses, encoding either Vpr (pHR-Vpr) or a control marker gene GFP (pHR-GFP). Cdc2 Tyr-15 phosphorylation levels were analyzed by Western blot in HeLa cells at forty-eight hours post-infection (FIG. 2). Cells infected with pHR-VPR (FIG. 2, lane 2), had increased levels of Cdc2 Tyr15 phosphorylation when compared to either mock-infected (FIG. 2, lane 1) or pHR-Vpr infected (FIG. 2, lane 3) cells. Caffeine decreased Cdc2 Tyr-15 phosphorylation in pHR-VPR infected cells (FIG. 2, lane 4). As an additional negative control, taxol treatment was used to arrest upon entry into mitosis. The arrest is concomitant with the presence of active Cdc2 kinase, which is not phosphorylated at Tyr-15 (FIG. 2, lane 5). As a positive control, doxorubicin treatment was used. Doxorubicin is a genotoxic agent that intercalates into DNA and induces G₂ arrest via Cdc2 Tyr-15 phosphorylation. Treatment with doxorubicin induced levels of Tyr-15 phosphorylation that were similar to those induced by Vpr expression (FIG. 2, lane 6). The increase of the Cdc2 phosphorylation on Tyr15 is likely due to the activity of checkpoint proteins involved in the DNA damage responsive G2 checkpoint.

### Example 2

### ATR Function is Required for Induction of Vpr-induced G₂ Arrest

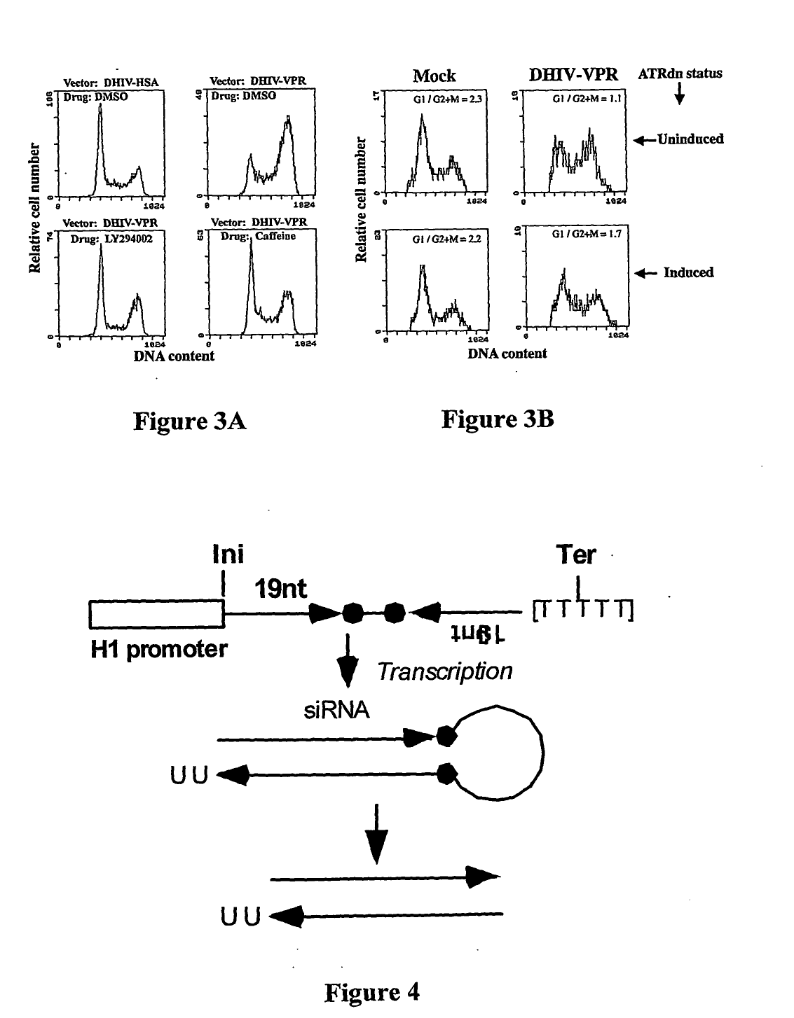

Caffeine is a radiosensitizing agent that inhibits PI3K-like protein kinase ATM and ATR function. The drug blocks the G₂ arrest induced by both genotoxic agents and Vpr. However, it has remained unclear whether the effect of caffeine is due to the inhibition of ATM and ATR. Another drug, LY294002, is a an inhibitor of PI3K family members (Smith et al., “DNA-Dependent Protein Kinase and Related Proteins,” Biochem Soc Svmp. 64:91-104 (1999); Vlahos et al., “A Specific Inhibitor of Phosphatidylinositol 3-Kinase, 2-(4-Morpholinyl)-8-Phenyl4H-1-Benzopyran-4-One (LY294002),” J. Biol. Chem. 269:5241-5248 (1994), each of which is hereby incorporated by reference in its entirety). To explore the potential role of the PI3K-like family members in the Vpr-induced arrest, the cell cycle profiles of Vpr-expressing cells was examined in the presence of LY294002. HeLa cells were treated with either 50 μM LY294002 in DMSO, or with 0.1% DMSO alone, and infected with either pHR-VPR or pHR-GFP. Thirty-six hours after infection, the cell cycle profiles were analyzed by flow cytometry (FIG. 3, panel A). Addition of LY294002 alleviated Vpr-induced G₂ arrest. In a previous study, Bartz et al. tested whether ATM −/−(AT) cell lines were able to arrest in response to Vpr (“Human Immunodeficiency Virus Type 1 Cell Cycle Control: Vpr is Cytostatic and Mediates G2 Accumulation by a Mechanism Which Differs From DNA Damage Checkpoint Control,” J. Virol. 70:2324-2331 (1996), which is hereby incorporated by reference in its entirety). Bartz et al. demonstrated that AT cells arrest with indistinguishable kinetics as ATM +/+cells. Subsequent to the experiments by Bartz et al., a new human PI3K-like protein, ATR, was identified (Bentley et al., “The Schizosaccharomyces Pombe rad3 Checkpoint Gene,” EMBO J. 15:6641-6651 (1996), which is hereby incorporated by reference in its entirety). Cliby et al. described a kinase deficient ATR that carries an Asp-2475 to Ala mutation within the catalytic domain of the protein (Cliby et al., “Overexpression of a Kinase-Inactive ATR Protein Causes Sensitivity to DNA-Damaging Agents and Defects in Cell Cycle Checkpoints,” EMBO J. 17:159-169 (1998), which is hereby incorporated by reference in its entirety). This ATR mutant, termed ATRkd (kinase-deficient), is defective in autophosphorylation and, when expressed in mammalian cells, acts as a dominant-negative regulator of wild-type ATR. U2OS7/ATRkd is a human fibroblast cell line that was stably transduced with a tetracycline-inducible ATRkd gene (Nghiem et al., “ATR inhibition Selectively Sensitizes GI Checkpoint-Deficient Cells to Lethal Premature Chromatin Condensation,” Proc Natl Acad Sci USA 98:9092-9097 (2001); Nghiem et al., “ATR is Not Required for p53 Activation but Synergizes With p53 in the Replication Checkpoint,” J. Biol. Chem. 15:15 (2001), each of which is hereby incorporated by reference in its entirety).

The U2OS/ATRkd cells were used to further investigate the role of ATR in Vpr-induced G₂ arrest (FIG. 3 B). Expression of ATRkd was induced by addition of 2 μM doxycycline for forty-eight hours. Following doxycycline induction, the cells were infected with VPR or with control viruses or infected with DHIV-VPR. The cell cycle profiles of the infected cells was then examined. Forty-eight hours after infection, uninduced ATRkd cells displayed a normal cell cycle profile (indicative of lack of G₂ arrest) when not infected and a typical accumulation in G2 (indicative of G₂ arrest) when infected with DHIV-VPR (FIG. 3, panel B). Therefore, U2OS/ATRkd cells, in the absence of ATRkd induction, are sensitive to the cytostatic effect of Vpr. After induction of ATRkd expression with doxycycline, mock-infected cells displayed a normal cell cycle profile, but were significantly less sensitive to Vpr-induced G₂ arrest when induced. Consistent with prior observations ATRkd overexpression also caused a reduction of the doxorubicin-induced G2 arrest. It is likely that doxorubicin causes a number of genomic abnormalities, activating both ATR and ATM, while Vpr-induced checkpoint requires ATR. To rule out a possibility that these observations may be specific to U2OS, an additional ATRkd expressing cell line, GM847, was also used. The GM847-ATRkd cell line becomes resistant to Vpr-induced G2 arrest upon ATRkd expression. Consistent with prior observations, the p53 status of the cells does not appear to influence Vpr-induced G2 arrest. U2OS contains wild-type p53 while GM847 cells are transformed with SV40 large T antigen, which blocks p53 function.

While overexpression of ATRkd has been used to study ATR function, it remains formally possible that ATRkd dominant negative affects (inhibits) the function of proteins other than ATR and, therefore, may also inhibit other checkpoint proteins. To rule out this possibility, an RNAi-mediated knockdown was used to further study the effect of ATR on Vpr-induced G2 arrest. RNA interference (RNAi) is a recently described mechanism utilized by eukaryotic cells to downregulate the steady-state levels and/or the translation of specific mRNAs (Elbashir et al., “Duplexes of 21-Nucleotide RNAs Mediate RNA Interference in Cultured Mammalian Cells,” Nature 411:494-498 (2001); Lee et al., “An Extensive Class of Small RNAs in Caenorhabditis elegans,” Science 294:862-864 (2001); Reinhart et al., “The 21-Nucleotide let-7 RNA Regulates Developmental Timing in Caenorhabditis Elegans,” Nature 403:901-906 (2000), each of which is hereby incorporated by reference in its entirety). RNAi is accomplished by short (21-22 nt) double-stranded RNA oligonucleotides, short interfering RNAs or siRNAs, that are specific for the targeted mRNA. This observation has been used to target heterologous mRNAs through changes in the sequence of the RNA oligonucleotides.

ATR was targeted using a novel plasmid construct that directs expression of siRNAs from an RNA polymerase III-specific promoter as shown in FIG. 4. In this system, siRNAs are produced as single-stranded hairpins that are processed by dicer to produce the mature and active double-stranded RNA oligonucleotides (Brummelkamp et al., “A System for Stable Expression of Short Interfering RNAs in Mammalian Cells,” Science 21:21 (2002), which is hereby incorporated by reference in its entirety).

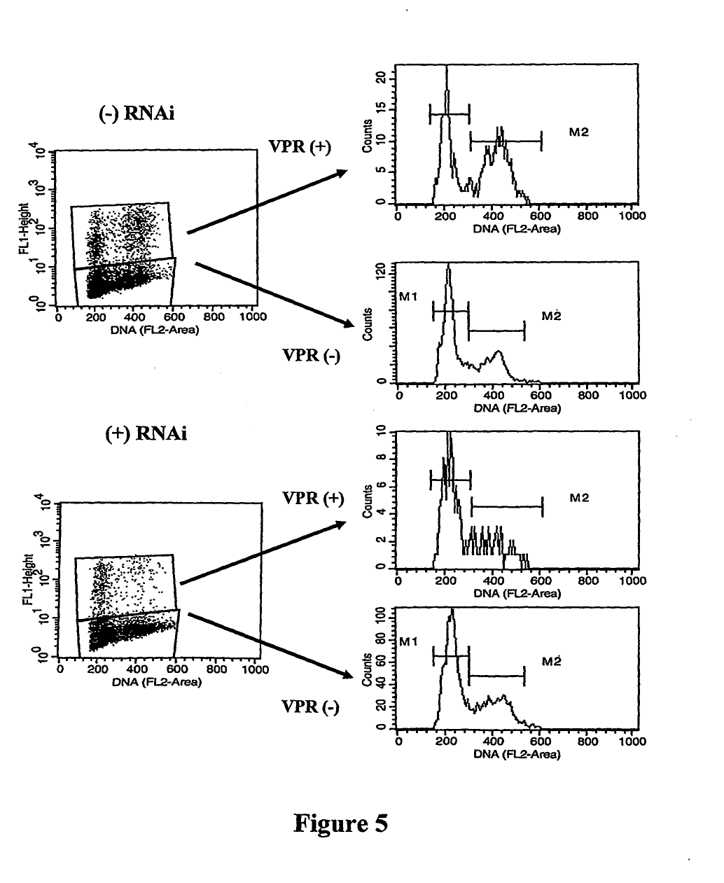

ATR target sequences were: AACCTCCGTGATGTTGCTTGA(SEQ ID NO: 4, target underlined) and AAGCCATGAGCGCAAAGGCAG(SEQ ID NO: 5, target underlined). siRNA targeted to these sequences were SEQ ID NOs: 1 and 2, respectively. Transduction of Vpr in the ATR specific-RNAi transfected cells (RNAi [+]) yielded a significantly attenuated G2 arrest as compared with HeLa cells transfected with empty vector (RNAi [−]) (FIG. 5).

### Example 3

### Vpr Induces ATR-dependent Chk1 Phosphorylation

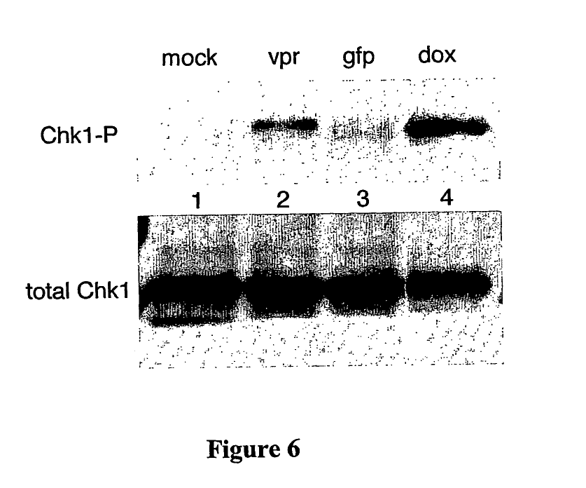

Chk1 is a direct target for ATR in response to DNA damage. ATR phosphorylates Chk1 on Ser345, resulting in increased Chk1 activity. Therefore, it was expected that Vpr-induced ATR activation would result in phosphorylation of Chk1 on Serine 345. HeLa cells were infected with either pHR-VPR or pHR-GFP, and forty-eight hours post-infection the phosphorylation status of Chk1 was analyzed by Western blot (FIG. 6). Mock-infected (FIG. 6, lane 1) and pHR-GFP-infected (FIG. 6, lane 2) cells only revealed a faint band corresponding to Chk1-P. However, cells infected with pHR-VPR (FIG. 6, lane 3) or treated with doxorubicin (FIG. 6, lane 4) displayed a significant amount of Chk1-P. Inhibition of ATR and ATM function with caffeine resulted in a significant decrease of Chk1 phosphorylation.

### Example 4

### Inhibition of Chk1 and Related Kinases by UCN-01 Results in Alleviation of the Vpr-induced G2 Arrest and Cdc2 Hyperphosphorylation on Tyr15

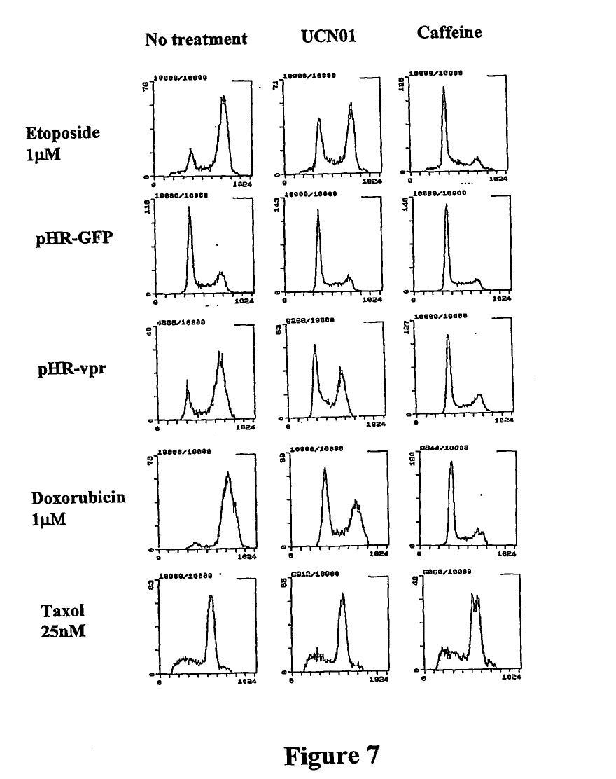

UCN-01 is a radiosensitizing agent that targets Chk1 , Chk2, and the Cdc25C-related kinase, c-Tak. However, the UCN-01 ID₅₀for Chk2 is about twenty fold greater than its ID₅₀ for Chk1 (Busby et al., “The Radiosensitizing Agent 7-Hydroxystaurosporine (UCN-01) Inhibits the DNA Damage Checkpoint Kinase hChk1,” Cancer Res. 60:2108-2112 (2000), which is hereby incorporated by reference in its entirety). Tests were performed to determine whether inhibition of the kinases would result in reduction of G2 arrest-induced by Vpr. HeLa cells were treated with either 2.5 mM caffeine, which inhibits ATR and ATM/Rad3 but not chk2; or 200 nM UCN-01, a concentration that is sufficient to completely inhibit Chk1 and c-TAK, but not Chk2 (Busby et al., “The Radiosensitizing Agent 7-Hydroxystaurosporine (UCN-01) Inhibits the DNA Damage Checkpoint Kinase hChk1,” Cancer Res. 60:2108-2112 (2000), which is hereby incorporated by reference in its entirety). Incubation with UCN-01 resulted in dramatic reduction of Vpr-induced G2 arrest, consistent with observations that UCN-01 reduced doxorubicin-induced G2 arrest (FIG. 7). UCN-01, however, did not affect the taxol-induced M phase arrest (FIG. 7).

### Example 5

### Potential Role of Rad17 in Vpr-induced G2 Arrest

It was hypothesized that similar to DNA-damage induced checkpoint activation, Vpr-induced G₂ arrest would require not only the activity of ATR, but also the ATR partners in the damage recognition and signaling. Rad17 is a RepC-like protein that is required for the ATR induced checkpoint activation (Bao et al., “ATR/ATM-Mediated Phosphorylation of Human Rad17 is Required for Genotoxic Stress Responses,” Nature 411:969-974 (2001), which is hereby incorporated by reference in its entirety) and regulates ATR substrate selection (Zou et al., “Regulation of ATR Substrate Selection by Rad17-Dependent Loading of Rad9 Complexes onto Chromatin,” Genes Dev. 16:198-208 (2002), which is hereby incorporated by reference in its entirety). Rad17 association with chromatin facilitates chromatin association of the PCNA-like complex formed by Rad9, Hus1, and Rad1, which promotes checkpoint induction (Roos-Mattjus et al., “Genotoxin-Induced Rad9-Hus1-Rad1 (9-1-1) Chromatin Association is an Early Checkpoint Signaling Event,” J. Biol. Chem. 277:43809-43812 (2002), which is hereby incorporated by reference in its entirety). Using RNAi, tests were performed to determine whether Rad17 maybe involved in the Vpr-induced checkpoint. The expression system described in FIG. 4 was used to express a Rad17-specific siRNA.

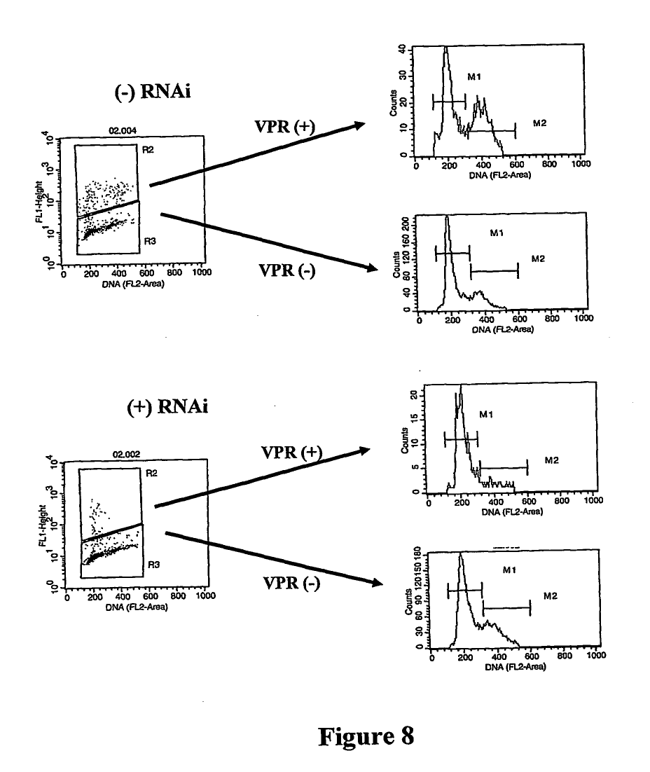

The Rad17 target sequence was AACAGACTGGGTTGACCCATC(SEQ ID NO: 6, target underlined). siRNA targeted to this sequence was SEQ ID NO: 3 (see Zou et al., “Regulation of ATR Substrate Selection by Rad17-Dependent Loading of Rad9 Complexes onto Chromatin,” Genes Dev. 16:198-208 (2002), which is hereby incorporated by reference in its entirety). Expression of a Rad17-specific siRNA, but not empty vector, eliminated the G2 arrest induce by Vpr expression (compare (−)RNAi/VPR(+) with (+)RNAi/VPR(+) in FIG. 8).

### Example 6

### Vpr Uses the Saccharomyces cerevisae ATR homolog, Mec1, to Induce G2 Arrest

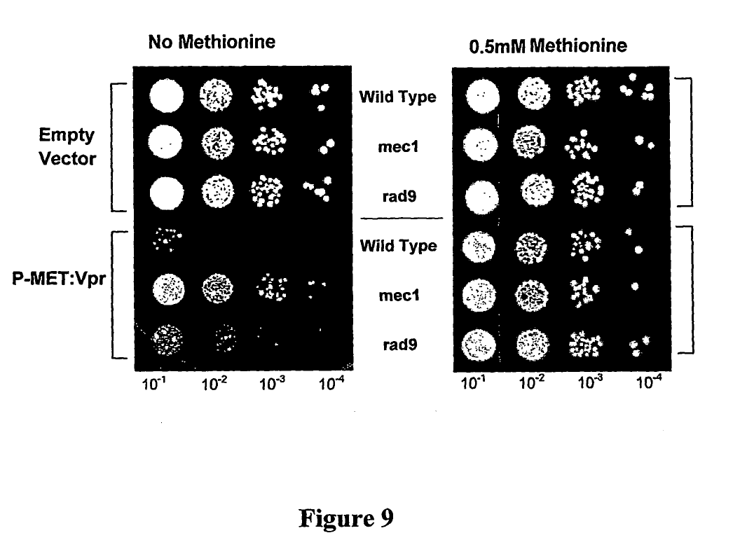

The biology of HIV-1 Vpr was tested to determine whether it could be studied in the budding yeast, Saccharomyces cerevisae. The S. cerevisiae expression vector for Vpr, denominated P-MET:Vpr, along with the empty vector control, M3885, were prepared. Transformation of P-MET:Vpr followed by methionine induction led to cell cycle arrest in G2 as evidenced by the formation of typical “dumbbell”-shaped cells in the budding yeast. A null mutant of mec1, mec1-A401 in the A364a background was analyzed, along with the mec1 wild-type counterpart of the same background, for growth defects upon induction of Vpr (FIG. 9). The growth of transformed strains was assayed by the spot-dilution method using 10-fold dilutions. Uninduced cultures or cultures transformed with empty vector demonstrated normal colony-formation potentials. The growth of wild-type yeast, however, was dramatically reduced by the presence of Vpr in the induced culture. This growth defect was largely eliminated in the Mec1 -A401 mutant strain. The elimination of the growth defect was not complete, as evidenced by the relatively small size of the vpr-expressing colonies.

In humans and yeast, ATR and Mec1 act in concert with Rad9, which forms a Rad9-Hus1-Rad1 ternary complex (Zou et al., “Regulation of ATR Substrate Selection by Rad17-Dependent Loading of Rad9 Complexes onto Chromatin,” Genes Dev. 16:198-208 (2002), which is hereby incorporated by reference in its entirety). In parallel with the above experiments, a Rad9 knockout mutant in S. cerevisiae, background A364a, appeared to moderately compensate for the growth defect induced by Vpr.

### Discussion of Examples 1-6

Using pharmacological, dominant negative regulator, and genetic approaches, the experimental results demonstrate that ATR activity is required for the induction of the Vpr-induced G2 arrest. Similar to the DNA-damage checkpoint, ATR activation leads to the phosphorylation of Chk1 and the induction of Cdc2 hyperphosphorylation. These observations, taken together, suggest the regulation of the transition between G₂ and M by Vpr is similar to the type induced by DNA damaging agents. The checkpoint induced by Vpr is highly conserved between yeast and humans and requires the activity of both Rad17 and Rad9 for full activation.

The experimental results presented did not address whether Vpr actually causes DNA damage or, alternatively, generates a signal that “mimics” DNA damage by activating one of the DNA damage sensors. Nevertheless, observations suggest at least some difference between the DNA-damage and Vpr-induced checkpoint activation exists. Inhibition of the checkpoint proteins in the context of DNA damage usually results in increase of apoptosis. In contrast, inhibition of the Vpr-induced checkpoint by caffeine actually results in a decrease of apoptosis (Zhu et al., “Comparison of Cell Cycle Arrest, Transactivation, and Apoptosis Induced by the Simian Immunodeficiency Virus SIVagm and Human Immunodeficiency Virus Type 1 vpr Genes,” J. Virol. 75:3791-3801 (2001), which is hereby incorporated by reference in its entirety). Surprisingly, Chk1 knockdown yields a different result. RNAi-mediated Chk1 knockdown resulted in the dramatic increase in the Vpr-induced apoptosis, suggesting that the activity of some, but not other checkpoint proteins is required for the survival of the Vpr-infected cells, at least in the short term. In the process of elucidating the mechanism of the Vpr-induced checkpoint activation, a yeast-based system was developed that can prove useful in further studies of the Vpr-biology. The mechanism of the Vpr-induced growth arrest in the budding yeast appears to be remarkably similar to the one induced in human cells. Previously, attempts have been made to study Vpr-biology in the fission yeast. In that system, neither the knockout of ATR/ATM homologue Rad3 nor chk1 and chk2 homologues resulted in the reduction of the Vpr-induced growth defect (Elder et al., “Cell Cycle G2 Arrest Induced by HIV-1 Vpr in Fission Yeast (Schizosaccharomyces pombe) is Independent of Cell Death and Early Genes in the DNA Damage Checkpoint,” Virus Res. 68(2):161-173 (2000), which is hereby incorporated by reference). It is possible that an alternative DNA-damage responsive system is activated in the fission yeast. The Rad3 mutations in the fission yeast are viable (Bentley et al., “The Schizosaccharomyces pombe rad3 Checkpoint Gene,” EMBO J. 15(23):6641-6651 (1996), which is hereby incorporated by reference in its entirety), while the Mec1 mutations in the fission yeast and ATR in human cells are lethal, suggesting an incomplete homology between the fission yeast Rad3 and ATR/Mec1 systems.

A recent report (de Noronha et al., “Dynamic Disruptions in Nuclear Envelope Architecture and Integrity Induced by HIV-1 Vpr,” Science 294:1105-1108 (2001), which is hereby incorporated by reference in its entirety) has demonstrated that Vpr induces defects in nuclear lamin structure and consequent nuclear herniation with chromatin structure alterations. The authors have suggested that Vpr-induced chromatin structure alteration may lead to incomplete replication of DNA during S phase. ATR has recently emerged as a key sensor of incomplete replication status of mammalian cells (Cliby et al., “S Phase and G2 Arrests Induced by Topoisomerase I Poisons are Dependent on ATR Kinase Function,” J. Biol. Chem. 277:1599-1606 (2002); Guo et al., “Requirement for ATR in Phosphorylation of Chk1 and Cell Cycle Regulation in Response to DNA Replication Blocks and UV-Damaged DNA in Xenopus Egg Extracts,” Genes Dev. 14:2745-2756 (2000); Hekmat-Nejad et al., “Xenopus ATR is a Replication-Dependent Chromatin-Binding Protein Required for the DNA Replication Checkpoint,” Curr Biol. 10:1565-1573 (2000); Tibbetts et al., “Functional Interactions Between BRCA1 and the Checkpoint Kinase ATR During Genotoxic Stress,” Genes Dev. 14:2989-3002 (2000), each of which is hereby incorporated by reference in its entirety) and, therefore, ATR is likely to mediate the cell cycle arrest caused by Vpr.

In view of the above findings, yet without being bound by theory, the following model for the signaling induced by Vpr can be proposed. Via interaction with lamins, Vpr induces alterations in the chromatin structure, which may lead to stalled replication. The previous alterations in chromatin structure and replication are sensed by ATR, which, in turn, activates Chk1 . Further activation of the ATR/Chk1 cascade leads to inhibition of Cdc2, the key regulator of the G₂/M transition. Likely candidates as the immediate inhibitors of Cdc2 may be Cdc25C (Re et al., “Human Immunodeficiency Virus Type 1 Vpr Arrests the Cell Cycle in G2 by Inhibiting the Activation of p34cdc2-Cyclin B,” J. Virol. 69:6859-6864 (1995), which is hereby incorporated by reference in its entirety) and Weel (Elder et al., “HIV-1 Vpr Induces Cell Cycle G2 Arrest in Fission Yeast (Schizosaccharomyces pombe) Through a Pathway Involving Regulatory and Catalytic Subunits of PP2A and Acting on Both Weel and Cdc25,” Virology 287:359-370 (2001); Masuda et al., “Genetic Studies with the Fission Yeast Schizosaccharomyces pombe Suggest Involvement of weel, ppa2, and rad24 in Induction of Cell Cycle Arrest by Human Immunodeficiency Virus Type 1 Vpr,” J. Virol. 74:2636-2646 (2000), each of which is hereby incorporated by reference in its entirety).

Although the invention has been described in detail for the purposes of illustration, it is understood that such detail is solely for that purpose, and variations can be made therein by those skilled in the art without departing from the spirit and scope of the invention which is defined by the following claims.

What is claimed is:

1. A method of inhibiting HIV replication comprising:
contacting a cell susceptible to HIV infection with an effective amount of an inhibitor of ATR or Rad17, or an inhibitor of an ATR-controlled pathway, under conditions effective to inhibit HIV replication in the cell.

2. The method according to claim 1, wherein the cell is a CD4-expressing cell.

3. The method according to claim 2, wherein the CD4-expressing cell is a T cell or a macrophage.

4. The method according to claim 1, wherein said contacting is carried out under conditions effective to inhibit G2 cell cycle arrest that is normally induced by HIV infection of the cell, wherein inhibition of G2 cell cycle arrest allows cell cycle progression to occur, thereby inhibiting HIV infectivity.

5. The method according to claim 1, wherein the HIV is HIV-1 or HIV-2.

6. The method according to claim 1, wherein the inhibitor of ATR is 2-(4-morpholinyl)-8-phenyl4H-1-benzopyran-4-one.

7. The method according to claim 1, wherein the inhibitor of ATR is an inhibitor of ATR expression.

8. The method according to claim 7, wherein the inhibitor of ATR expression is an inhibitory RNA molecule.

9. The method according to claim 8, wherein the inhibitory RNA molecule comprises less than about 30 nucleotides.

10. The method according to claim 8, wherein the inhibitory RNA molecule comprises the nucleotide sequence of SEQ ID NO: 1 or SEQ ID NO: 2.

11. The method according to claim 1, wherein the inhibitor of the ATR-controlled pathway is an inhibitor of Chk-1.

12. The method according to claim 1, wherein the inhibitor of Chk-1 is 7-hydroxystaurosporine.

13. The method according to claim 1, wherein the inhibitor of Rad17 is an inhibitor of Rad17 expression.

14. The method according to claim 13, wherein the inhibitor of Rad17 expression is an inhibitory RNA molecule.

15. The method according to claim 14, wherein the inhibitory RNA molecule comprises less than about 30 nucleotides.

16. The method according to claim 14, wherein the inhibitory RNA molecule comprises the nucleotide sequence of SEQ ID NO: 3.

17. The method according to claim 1, wherein the cell is ex vivo or in vivo.

18. A method of treating or preventing an HIV infection comprising:
administering to a patient an amount of an inhibitor of ATR or Rad17, or an inhibitor of an ATR-controlled pathway, which is effective to inhibit HIV replication in a cell susceptible to HIV infection.

19. The method according to claim 18, wherein the cell is a CD4-expressing cell.

20. The method according to claim 19, wherein the CD4-expressing cell is a T cell or a macrophage.

21. The method according to claim 18, wherein said administering is carried out orally, parenterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes, or by transdermal delivery.

22. The method according to claim 18, wherein said administering occurs prior to HIV exposure.

23. The method according to claim 18, wherein said administering occurs after HIV exposure.

24. The method according to claim 18, wherein said administering is effective to inhibit G2 cell cycle arrest in the cell following HIV infection of the cell, wherein inhibition of G2 cell cycle arrest allows cell cycle progression to occur, thereby inhibiting HIV infectivity.

25. The method according to claim 18, wherein the HIV is HIV-1 or HIV-2.

26. The method according to claim 18, wherein the inhibitor of ATR is 2-(4-morpholinyl)-8-phenyl-4H-1-benzopyran-4-one.

27. The method according to claim 18, wherein the inhibitor of ATR is an inhibitor of ATR expression.

28. The method according to claim 27, wherein the inhibitor of ATR expression is an inhibitory RNA molecule.

29. The method according to claim 28, wherein the inhibitory RNA molecule comprises less than about 30 nucleotides.

30. The method according to claim 28, wherein the inhibitory RNA molecule comprises the nucleotide sequence of SEQ ID NO: 1 or SEQ ID NO:2.

31. The method according to claim 18, wherein the inhibitor of Rad17 is an inhibitor of Rad17 expression.

32. The method according to claim 31, wherein the inhibitor of Rad17 expression is an inhibitory RNA molecule.

33. The method according to claim 32, wherein the inhibitory RNA molecule comprises less than about 30 nucleotides.

34. The method according to claim 32, wherein the inhibitory RNA molecule comprises the nucleotide sequence of SEQ ID NO: 3.

35. The method according to claim 18, wherein the inhibitor of the ATR-controlled pathway is an inhibitor of Chk-1.

36. The method according to claim 18, wherein the inhibitor of Chk-1 is 7-hydroxystaurosporine.

37. A method of inhibiting the activity of a viral protein R (Vpr) comprising:
contacting ATR or Rad17 with an effective amount of an inhibitor of ATR or Rad17, respectively, or contacting a component of an ATR-controlled pathway with an inhibitor thereof, under conditions effective to inhibit viral protein R activity which occurs via a pathway under regulatory control of ATR or Rad17.

38. The method according to claim 37, wherein the Vpr is HIV-1 Vpr or HIV-2 Vpr.

39. The method according to claim 37, wherein the inhibitor of ATR is 2-(4-morpholinyl)-8-phenyl-4H-1-benzopyran-4-one.

40. The method according to claim 37, wherein the inhibitor of the ATR-controlled pathway is an inhibitor of Chk-1.

41. The method according to claim 40, wherein the inhibitor of Chk-1 is 7-hydroxystaurosporine.

42. The method according to claim 37, wherein the inhibitor of ATR is an inhibitor of ATR expression.

43. The method according to claim 42, wherein the inhibitor of ATR expression is an inhibitory RNA molecule.

44. The method according to claim 43, wherein the inhibitory RNA molecule comprises less than about 30 nucleotides.

45. The method according to claim 43, wherein the inhibitory RNA molecule comprises the nucleotide sequence of SEQ ID NO: 1 or SEQ ID NO: 2.

46. The method according to claim 37, wherein the inhibitor of Rad17 is an inhibitor of Rad17 expression.

47. The method according to claim 46, wherein the inhibitor of Rad17 expression is an inhibitory RNA molecule.

48. The method according to claim 47, wherein the inhibitory RNA molecule comprises less than about 30 nucleotides.

49. The method according to claim 47, wherein the inhibitory RNA molecule comprises the nucleotide sequence of SEQ ID NO: 3.

50. The method according to claim 37, wherein the Vpr is in vitro or in vivo.

51. A method of treating a latent HIV infection in a patient comprising:
administering to a patient a first agent that activates latently infected patient cells to induce HIV Vpr expression; and
administering to the patient a second agent that activates the ATR-controlled pathway.

52. The method according to claim 51, wherein the agent that activated latently infected patient cells is a cytokine or a mitogenic stimulus.

53. The method according to claim 51, wherein the activator of the ATR-controlled pathway is a genotoxic agent.

54. The method according to claim 53, wherein the genotoxic agent is doxorubicin, etoposide, or radiation.

55. The method according to claim 51, wherein said administering the first agent occurs prior to said administering the second agent

56. The method according to claim 51, wherein said administering the second agent occurs prior to said administering the first agent.

57. The method according to claim 51, wherein said administering the first and second agents occurs simultaneously.

58. An inhibitory RNA molecule, the inhibitory RNA molecule binding to mRNA encoding ATR under conditions effective to inhibit expression of the mRNA.

59. The inhibitory RNA molecule according to claim 58, wherein the inhibitory RNA molecule comprises less than about 30 nucleotides.

60. The inhibitory RNA molecule according to claim 58, wherein the inhibitory RNA molecule binds to mRNA encoding ATR

61. The inhibitory RNA molecule according to claim 60, wherein the inhibitory RNA molecule comprises the nucleotide sequence of SEQ ID NO: 1 or SEQ ID NO: 2.

62. The inhibitory RNA molecule according to claim 58 in isolated form.

63. A DNA molecule encoding the inhibitory RNA molecule according to claim 58.

64. A DNA construct comprising the DNA molecule of claim 63 operably coupled at the 5′ end thereof to a promoter-effective DNA molecule and operably coupled at the 3′ end thereof to a transcription termination signal.

65. An expression vector comprising the DNA construct of claim 64.

66. A host cell transformed with the DNA construct according to claim 64.

67. The host cell according to claim 66, wherein the host cell is CD4⁺.

68. The host cell according to claim 66, wherein the host cell is a T cell or a macrophage.

69. A host cell that contains therein an inhibitory RNA molecule according to claim 58.
